(19) United States
(12) Patent Application Publication
(10) Pub. No.: US 2011/0190348 A1
(43) Pub. Date: Aug. 4, 2011

(54) METHODS FOR TREATING CNS DISORDERS

(76) Inventor: Pradeep Banerjee, Hillsborouch, NJ (US)

(21) Appl. No.: 13/059,944

(22) PCT Filed: Aug. 21, 2009
(86) PCT No.: PCT/US09/54569

§ 371 Date: Apr. 25, 2011

Related U.S. Application Data
(60) Provisional application No. 61/090,630, filed on Aug. 21, 2008.

(51) Int. Cl. A61K 31/454; A61P 25/28; A61P 25/24; A61P 25/22

(52) U.S. Cl. 514/321

(57) ABSTRACT

The present invention relates to methods for treating central nervous system disorders, such as Alzheimer's disease, anxiety and major depressive disorder, by administering piperidine derivatives, e.g., 2-[4-(4-fluoro-benzyl)-piperidine-1-yl]-2-oxo-N-(2-oxo-2,3-dihydro-benzoxazol-6-yl)acetamide, and pharmaceutically acceptable salts thereof.

### FIELD OF THE INVENTION

The present invention relates to methods for treating central nervous system disorders, such as Alzheimer's disease, anxiety and major depressive disorder, by administering piperidine derivatives, e.g., 2-[4-(4-fluoro-benzyl)-piperidine-1-yl]-2-oxo-N-(2-oxo-2,3-dihydro-benzoxazol-6-yl)acetamide, and pharmaceutically acceptable salts thereof.

### BACKGROUND OF THE INVENTION

Alzheimer's disease (AD) is a progressive neurodegenerative disorder, which primarily affects the elderly. Alzheimer's disease is characterized by two major pathologic observations in the brain: neurofibrillary tangles and beta amyloid (or neuritic) plaques, comprised predominantly of an aggregate of a peptide fragment know as Aβ. Individuals with AD exhibit characteristic beta-amyloid deposits in the brain (beta amyloid plaques) and in cerebral blood vessels (beta amyloid angiopathy) as well as neurofibrillary tangles. Neurofibrillary tangles occur not only in Alzheimer's disease but also in other dementia-inducing disorders. On autopsy, large numbers of these lesions are generally found in areas of the human brain important for memory and cognition.

There are two forms of AD, early-onset and late-onset. Early-onset AD is rare, strikes susceptible individuals as early as the third decade, and is frequently associated with mutations in a small set of genes. Late onset, or spontaneous, AD is common, strikes in the seventh or eighth decade, and is a mutifactorial disease with many genetic risk factors. Late-onset AD is the leading cause of dementia in persons over the age of 65. Early in the disease, patients experience loss of memory and orientation. As the disease progresses, additional cognitive functions become impaired, until the patient is completely incapacitated. Therefore, there is an urgent need for pharmaceutical agents capable of slowing the progression of Alzheimer's disease and/or preventing it in the first place.

Mood disorders, of which major depressive disorder is the most common, affect one person in five during their lifetime. The World Health Organization estimates that depression is currently the fourth most important worldwide cause of disability-adjusted life year loss, and that it will become the second most important cause by 2020 (See, Science, 288, 39-40, 2000). Major depressive disorder is a serious mental disorder that profoundly affects an individual's quality of life. Unlike normal bereavement or an occasional episode of “the blues,” MDD causes a lengthy period of gloom and hopelessness, and may rob the sufferer of the ability to take pleasure in activities or relationships that were previously enjoyable. In some cases, depressive episodes seem to be triggered by an obviously painful event, but MDD may also develop without a specific stressor. Research indicates that an initial episode of depression is likely to be a response to a specific stimulus, but later episodes are progressively more likely to start without a triggering event. A person suffering major depression finds jobrelated responsibilities and such other tasks as parenting burdensome and carried out only with great effort. Mental efficiency and memory are affected, causing even simple tasks to be tiring and irritating. Sexual interest dwindles; many people with MDD become withdrawn and avoid any type of social activity. Even the ability to enjoy a good meal or a sound night's sleep is frequently lost; many depressed people report a chronic sense of malaise (general discomfort or unease). For some, the pain and suffering accompanying MDD becomes so unendurable that suicide is viewed as the only option; MDD has the highest mortality rate of any mental disorder.

The condition of an individual suffering from a major depressive disorder is sometimes complicated by the fact that the individual is also suffering from anxiety. Thus in addition to the symptoms of their depressive illness, the patient may show signs of excessive or uncontrolled worry, irritability, feelings of tension, fears, restlessness and insomnia, difficulty in concentrating, and multiple somatic complaints such as pains and aches, twitching, stiffness, myoclonic jerks, tinnitus, blurred vision, hot and cold flushes, etc., all of which add to the individual's social and occupational impairment.

Pharmaceutical treatment of depression is frequently inadequate, with many patients typically not achieving remission, even after several months of treatment. Further, there are high recurrence rates—approximately 85% of patients who achieve remission will suffer another episode of major depression. Finally, many currently available antidepressants are associated with side effects that lead some patients to stop taking their medications at risk of sinking back (further) into depression, and to morbidity in others. Thus, many of today's drugs are neither completely safe nor completely tolerable for many patients.

There is, therefore, an existing and continual need for new pharmaceuticals to treat conditions such as Alzheimer's disease, major depressive disorder and anxiety, where the pharmaceuticals are effective for a broader range of patients (particularly for patients resistant to available pharmaceuticals), that are safe and more tolerable, or that complement the efficacy of existing drugs.

### SUMMARY OF THE INVENTION

In one aspect, the present invention relates to methods of Alzheimer's disease comprising administering piperidine derivatives, such as 2-[4-(4-fluoro-benzyl)-piperidine-1-yl]-2-oxo-N-(2-oxo-2,3-dihydro-benzoxazol-6-yl)acetamide, and pharmaceutically acceptable salts thereof. In other embodiments, methods of treating major depressive disorder and anxiety are described.

### DETAILED DESCRIPTION OF THE INVENTION

In one aspect, the present invention relates to methods of treating a CNS disorder (e.g., Alzheimer's disease, major depressive disorder, anxiety) comprising administering to a patient in need thereof, a therapeutically effective amount of a compound of formula (I):

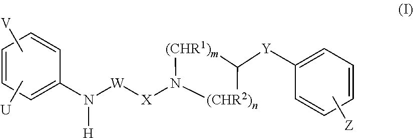

wherein

V and U are each independently

hydrogen, halogen, hydroxyl, cyano, nitro, amino, C₁-C₄ alkylamino optionally substituted by one or more halogen, arylamino optionally substituted by one or more halogen, aralkylamino optionally substituted by one or more halogen, C₁-C₄ alkylsulfonamido optionally substituted by one or more halogen, C₁-C₄ alkanoylamido optionally substituted by one or more halogen, arylsulfonamido, C₁-C₄ alkylsulfonyloxy, carboxyl, trifluoromethyl, trifluoromethoxy, C₁-C₄ alkyl-SO₂—NH—CH₂—, NH₂—(CH₂)₁₋₄—SO₂—NH—, NH₂—(CH₂)₁₋₄—(CO)—NH—, sulfamoyl [NH₂—SO₂-], formyl [—CHO], aminomethyl [—CH₂—NH₂], hydroxymethyl, C₁-C₄ alkyl, C₁-C₄ alkoxymethyl, halogenated methyl, tetrazolyl,

or C₁-C₄ alkoxy, C₁-C₄ alkoxycarbonyl, C₁-C₆ alkanoyloxy, phenyl or C₁-C₄ alkoxy, each of which is optionally substituted by an amino group, or

neighboring V and U groups, together with one or more identical or different additional heteroatoms and/or —CH═ and/or —CH₂— groups optionally form a substituted 4-7 membered homo- or heterocyclic ring (e.g., morpholine, pyrrole, pyrrolidine, oxo-pyrrolidine, thioxo-pyrrolidine, pyrazole, pyrazolidine, imidazole, imidazolidine, oxo-imidazole, thioxo-imidazole, imidazolidine, 1,4-oxazine, oxazole, oxazolidine, oxo-oxazolidine, thioxo-oxazolidine or 3-oxo-1,4-oxazine);

W and X are each independently —CO—, —CH₂— or —CH(C₁-C4 alkyl)-, with the proviso that W and X can not simultaneously be methylene;

Y is —O—, C₁-C₄ alkylene, C₁-C₄ alkynylene, cycloalkylene, aminocarbonyl, —NH—, —N(C₁-C₄ alkyl)-, —CH₂O—, —CH(OH)— or —OCH₂—;

Z is hydrogen, halogen, nitro, amino, C₁-C₄ alkyl, C₁-C₄ alkoxy, cyano, trifluoromethyl, hydroxyl or carboxy;

R¹ and R² are each independently hydrogen or alkyl, or R¹ and R² together form an optionally substituted C₁-C₃ bridge and

n and m independently are 0-3, with the proviso that n and m can not simultaneously be 0;

and pharmaceutically acceptable salts or solvates (e.g., hydrates) thereof, or solvates of pharmaceutically acceptable salts thereof;

with the further provisos that

when Z is hydrogen, Y is —CH₂—, m and n are 2, R¹ and R² are hydrogen, W is —CO—, X is —CH₂— and V is hydrogen, then U is other than a 4-bromo substituent, and

when Z is hydrogen, Y is —CH₂—, m and n are 2, R¹ and R² are hydrogen, W and X are —CO— and V is hydrogen, then U is other than a 4-carboxyl or 4-ethoxycarbonyl substituent.

In one embodiment, the compound of formula (I) is 2-[4-(4-fluoro-benzyl)-piperidine-1-yl]-2-oxo-N-(2-oxo-2,3-dihydro-benzoxazol-6-yl)acetamide (radiprodil), or a pharmaceutically acceptable salt thereof. The synthesis of radiprodil is described, for example, in U.S. Publication No. 2004/0157886.

In another embodiment, the present invention relates to a method of treating Alzheimer's disease comprising administering to a patient in need thereof a therapeutically effective amount of a compound of formula (I) (e.g., 2-[4-(4-fluoro-benzyl)-piperidine-1-yl]-2-oxo-N-(2-oxo-2,3-dihydro-benzoxazol-6-yl)acetamide) or a pharmaceutically acceptable salt thereof.

In yet another embodiment, the present invention relates to the treatment of depression (e.g., major depressive disorder) comprising administering to a patient in need thereof a therapeutically effective amount of a compound of formula (I) (e.g., 2-[4-(4-fluoro-benzyl)-piperidine-1-yl]-2-oxo-N-(2-oxo-2,3-dihydro-benzoxazol-6-yl)acetamide) or a pharmaceutically acceptable salt thereof.

In another embodiment, the present invention relates to the treatment of major depressive disorder with anxiety comprising administering to a patient in need thereof a therapeutically effective amount of a compound of formula (I) (e.g., 2-[4-(4-fluoro-benzyl)-piperidine-1-yl]-2-oxo-N-(2-oxo-2,3-dihydro-benzoxazol-6-yl)acetamide) or a pharmaceutically acceptable salt thereof.

In yet another embodiment, the present invention relates to the treatment of anxiety comprising administering to a patient in need thereof a therapeutically effective amount of a compound of formula (I) (e.g., 2-[4-(4-fluoro-benzyl)-piperidine-1-yl]-2-oxo-N-(2-oxo-2,3-dihydro-benzoxazol-6-yl)acetamide) or a pharmaceutically acceptable salt thereof.

In certain embodiments, the compound of formula (I) (e.g., 2-[4-(4-fluoro-benzyl)-piperidine-1-yl]-2-oxo-N-(2-oxo-2,3-dihydro-benzoxazol-6-yl)acetamide) is administered in an amount of between about 0.01 mg and about 150 mg, for example between about 5 mg and about 150 mg, such as between about 10 mg and about 150 mg.

In additional embodiments, the compound of formula (I) (e.g., 2-[4-(4-fluoro-benzyl)-piperidine-1-yl]-2-oxo-N-(2-oxo-2,3-dihydro-benzoxazol-6-yl)acetamide) is administered in an amount of about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

In another embodiment, the present invention relates to a method of treating Alzheimer's disease comprising administering to a patient in need thereof a therapeutically effective amount of radiprodil or a pharmaceutically acceptable salt thereof in the dosage amount from about 10 mg to about 150 mg to a patient in need thereof.

In yet another embodiment, the present invention relates to a method of treating Alzheimer's disease comprising administering to a patient in need thereof a therapeutically effective amount of radiprodil or a pharmaceutically acceptable salt thereof in the dosage amount of about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

In yet another embodiment, the present invention relates to the treatment of depression (e.g., major depressive disorder) comprising administering to a patient in need thereof a therapeutically effective amount of radiprodil or a pharmaceutically acceptable salt thereof in the dosage amount from about 10 mg to about 150 mg to a patient in need thereof.

In yet another embodiment, the present invention relates to the treatment of depression (e.g., major depressive disorder) comprising administering to a patient in need thereof a therapeutically effective amount of radiprodil or a pharmaceutically acceptable salt thereof in the dosage amount of about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

In another embodiment, the present invention relates to the treatment of major depressive disorder with anxiety comprising administering to a patient in need thereof a therapeutically effective amount of radiprodil or a pharmaceutically acceptable salt thereof in the dosage amount from about 10 mg to about 150 mg to a patient in need thereof.

In another embodiment, the present invention relates to the treatment of major depressive disorder with anxiety comprising administering to a patient in need thereof a therapeutically effective amount of radiprodil or a pharmaceutically acceptable salt thereof in the dosage amount of about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

In yet another embodiment, the present invention relates to the treatment of anxiety comprising administering to a patient in need thereof a therapeutically effective amount of radiprodil or a pharmaceutically acceptable salt thereof in the dosage amount from about 10 mg to about 150 mg to a patient in need thereof.

In yet another embodiment, the present invention relates to the treatment of anxiety comprising administering to a patient in need thereof a therapeutically effective amount of radiprodil or a pharmaceutically acceptable salt thereof in the dosage amount of about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

The desired dose may be administered as one or more daily sub dose(s) administered at appropriate time intervals throughout the day, or alternatively, in a single dose, for example, for morning or evening administration. For example, the daily dosage may be divided into one, into two, into three, or into four divided daily doses. In certain embodiments, the active ingredient is administered in one, two or three (e.g., three) divided daily doses.

The duration of the treatment may be decades, years, months, weeks, or days, as long as the benefits persist.

Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

In one embodiment, the pharmaceutically acceptable salt is a hydrochloride salt.

Some of the compounds useful in the present invention can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds. The use of such polymorphs is within the scope of the present invention.

Some of the compounds useful in the present invention can exist in different solvate forms. Solvates of the compounds of the invention may also form when solvent molecules are incorporated into the crystalline lattice structure of the compound molecule during the crystallization process. For example, suitable solvates include hydrates, e.g., monohydrates, dihydrates, sesquihydrates, and hemihydrates. The use of such solvates is within the scope of the present invention.

The compounds of formula (I) can be administered either alone as an active ingredient or as an additional ingredient of a pharmaceutically acceptable composition.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage forms is closely related to the therapeutic amounts of the compounds or compositions which are desirable and efficacious for the given treatment application.

Suitable dosage forms include but are not limited to oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterile administration, and other dosage forms for systemic delivery of active ingredients. Formulations suitable for oral administration are preferred.

Various solid oral dosage forms can be used for administering active ingredient including such solid forms as tablets, gelcaps, capsules, caplets, granules, lozenges and bulk powders. In such solid dosage forms the active ingredient is mixed with at least one inert, pharmaceutically acceptable carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quarternary ammonium salts, g) wetting agents such as, for example cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Compositions suitable for buccal or sublingual administration include tablets, lozenges and pastilles, wherein the active ingredient is formulated with a carrier such as sugar and acacia, tragacanth, or gelatin and glycerin.

The solid dosage forms of tablets, capsules, pills and granules can be prepared with coatings and shells such as enteric coatings and other coatings well known in the pharmaceutical formulating art. They may optionally contain opacifying agents and can also be of a composition that they release the crystalline compound of the present invention. In another embodiment of the present invention, radiprodil can be formulated in a time release capsules, tablets and gels which is also advantageous in the targeted release of the crystalline compound of the present invention.

Various liquid oral dosage forms can also be used for administering active ingredient, including aqueous and non-aqueous solutions, emulsions, suspensions, syrups, and elixirs. In addition to the active ingredient, the liquid dosage forms may contain inert diluents commonly used in the art such as, for example, water or other solvents, solubilizing agents and emulsifiers, for example ethyl alcohol, ethyl carbonate, ethyl acetate, propylene glycol, oils, fatty acid esters and mixtures thereof. Besides inert diluents, the oral compositions can also include adjuvants such as wetting agents, emulsifying and suspending agents, sweetening, flavoring and perfuming agents. Aerosol formulations typically comprise typically comprise a solution or fine suspension of the crystalline compound of the present invention in physiologically acceptable aqueous or non-aqueous solvent and are usually presented in single or multidose quantities in sterile form in a sealed container.

Injectable preparations of the present invention, for example, sterile injectable aqueous or oleaginous suspensions may be formulated according to the known art using suitable dispersing or wetting agents and suspending agents.

Suppositories for rectal administration of the active ingredient can be prepared by mixing the compound with a suitable excipient such as cocoa butter, salicylates and polyethylene glycols. Formulations for vaginal administration can be in the form of a pessary, tampon, cream, gel, past foam, or spray formula containing, in addition to the active ingredient, such suitable carriers as are known in the art.

For topical administration, the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the active ingredient can be administered by inhalation in the form of a powder (e.g., micronized) or in the form of atomized solutions or suspensions. The aerosol formulation can be placed into a pressurized acceptable propellant.

The invention also provides the use of compounds of formula (I) in the manufacture of a medicament for the treatment of conditions such as Alzheimer's disease, major depressive disorder and anxiety.

In one embodiment, the compositions of the present invention contain radiprodil between about 0.01% by weight and about 25%, between about 0.05% and about 25%, between about 0.1% and about 25%, between about 0.25% and about 25%, between about 0.5% and about 25%, between about 1% and about 25%, between about 2% and about 20%, between about 4% and about 18%, between about 6% and about 16%, between about 8% and about 14%, between about 10% and about 12% by weight of the pharmaceutically acceptable composition.

To prepare such pharmaceutical dosage forms, the active ingredient is typically mixed with a pharmaceutical carrier according to conventional pharmaceutical compounding techniques. The carrier may take a wide variety of forms depending on the form of preparation desired for administration.

In preparing the compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. Due to their ease in administration, tablets and capsules represent the most advantageous oral dosage unit form. If desired, tablets may be sugar coated or enteric coated by standard techniques.

For parenteral formulations, the carrier will usually comprise sterile water, though other ingredients, for example, ingredients that aid solubility or for preservation, may be included. Injectable solutions may also be prepared in which case appropriate stabilizing agents may be employed.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Treatment methods of the present invention using formulations suitable for oral administration may be presented as discrete units such as capsules, cachets, tablets, or lozenges, each containing a predetermined amount of the active ingredient as, for example, a powder or granules. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration usually comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations of this invention may further include one or more accessory ingredient(s) selected from diluents, buffers, flavoring agents, binders, disintegrants, surface active agents, thickeners, lubricants, preservatives (including antioxidants), and the like.

The formulations of the present invention can have immediate release, sustained release, delayed-onset release or any other release profile known to one skilled in the art.

The compound of formula (I) may be adjunctively administered in combination with additional active agents useful in the treatment of CNS disorders (e.g., Alzheimer's disease, major depressive disorder). For example, the compound of formula (I) may be administered in combination with, for example, an antidepressant (e.g., tricyclic antidepressant), selective serotonin reuptake inhibitor, norepinephrine reuptake inhibitor, norepinephrine-dopamine reuptake inhibitor, serotonin-norepinephrine reuptake inhibitor (e.g., SNRI), monoamine oxidase inhibitor, cholinesterase inhibitor, and combinations thereof.

Specific examples of compounds that can be administered with the compound of formula (I) include, but are not limited to, memantine, escitalopram, citalopram, milnacipran, donezepil, rivastigmine, galantamine, fluvoxamine, paroxetine, reboxetine, sertraline, amitriptyline, desipramine, nortriptyline, duloxetine, venlafaxine, mirtazepine, trazodone, bupropion and combinations thereof (including salts and/or solvates thereof).

For example, the compound of formula (I) (e.g., radiprodil) may be administered with memantine, or a pharmaceutically acceptable salt thereof (e.g., memantine hydrochloride) for the treatment of Alzheimer's disease.

In another example, the compound of formula (I) (e.g., radiprodil) may be administered with escitalopram, or a pharmaceutically acceptable salt thereof (e.g., escitalopram oxalate) for the treatment of depression (e.g., major depressive disorder).

In another example, the compound of formula (I) (e.g., radiprodil) may be administered with milnacipran, or a pharmaceutically acceptable salt thereof (e.g., milnacipran hydrochloride) for the treatment of depression (e.g., major depressive disorder).

By adjunctive administration is meant simultaneous administration of the compounds in the same-dosage form, simultaneous administration in separate dosage forms or separate administration of the compounds.

### DEFINITIONS

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans.

The terms “treat,” “treatment,” and “treating” refer to one or more of the following:
(a) relieving or alleviating at least one symptom of a disorder in a subject, including for example, diabetic neuropathic pan, post-herpetic neuralgia;
(b) relieving or alleviating the intensity and/or duration of a manifestation of a disorder experienced by a subject including, but not limited to, those that are in response to a given stimulus (e.g., pressure, tissue injury, cold temperature, etc.);
(c) arresting, delaying the onset (i.e., the period prior to clinical manifestation of a disorder) and/or reducing the risk of developing or worsening a disorder.

An “effective amount” means the amount of an active ingredient that, when administered to a patient (e.g., a mammal) for treating a disease, is sufficient to effect such treatment for the disease, or an amount that is sufficient for modulating an NMDA receptor (e.g., NR2B receptor) to achieve the objectives of the invention. The “effective amount” will vary depending on the compound, the disease and its severity and the age, weight, responsiveness, etc., of the patient to be treated.

A subject or patient in whom administration of the therapeutic compound is an effective therapeutic regimen for a disease or disorder is preferably a human, but can be any animal, including a laboratory animal in the context of a trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

### Example 1

The aim of this study was to evaluate the antidepressant activity of radiprodil. The forced swimming test is a behavioral assay that can be used to predict antidepressant efficacy of drugs in humans.

### Animals

Male NMRI mice weighing 24-26 g were used. The animals were kept in polycarbonate cages in a thermostatically controlled room at 24+2° C. and at a relative humidity (RH) of 50±10%. The room was artificially illuminated from 6 am to 6 pm and the mice were given commercial pellet rat-mouse feed, autoclaved at 105° C. and sterile filtered tap water, ad libitum.

### Dosing

An aqueous solution of radiprodil, in the form of a complex with heptakis(2,6-di-O-methyl)-β-cyclodextrin(DIMEB), was administered orally at doses of 2.5, 5 and 10 mg/kg (calculated for non-complexed radiprodil). DIMEB-80 (heptakis(2,6-di-O-methyl)-β-cyclodextrin with an isomeric purity >80%) was used as the control, dissolved in water at a concentration of 7 mg/ml. All solutions were administered at a volume of 10 mL/kg.

### Procedures

Forced Swimming Test

The forced swimming test (FST) was measured in glass cylinders (height: 185 cm, diameter 14 cm) containing 12 cm of water, maintained at 23-25° C. Each test group consisted of 10 mice. 60 minutes after oral administration of the test compound or vehicle, the mice were individually placed in the glass cylinder for 6 minutes. The duration of immobility was recorded using a stopwatch. The mouse was judged to be immobile if it was floating in an upright position, and made only small movements to keep its head above water.

Spontaneous Locomotor Activity

Spontaneous locomotor activity was measured in a four-channel activity monitor manufactured by Farmakotechnika (Hungary). The apparatus consisted of acrylic cages (43 cm×43 cm×32 cm) equipped with 2×16 pairs of photocells along the bottom axis of the cage. An additional array of photocells (16 pairs) was placed along two opposite sides of the cage at a height of 10 cm in order to detect rearing responses. The photocell beam, when broken, signaled a count, which was then recorded by a computer.

Each group consisted of 10 animals. Thirty minutes after oral administration of the test compound or vehicle, the animals were individually placed in one of four cages for 1 hour. The horizontal and vertical movements were determined as the number of beam interruptions collected in 15 minute intervals throughout the 1 hour period.

### Data Analysis

In the forced swimming test, the mean±standard error on the mean (s.e.m) immobility time in seconds was measured. For comparison of these results with the spontaneous locomotor activity data, an “FST-activity” variable was also calculated for each animal by subtracting the immobility time from the total time (i.e., 360 seconds—immobility time). The mean (±s.e.m) “FST-activity” value was also calculated for each group and submitted to the same data analysis process as the immobility data.

In the spontaneous locomotor activity test, the mean (±s.e.m) value of one hour horizontal activity data (in counts) of each group was calculated.

The significance of the drug effect was determined by ANOVA followed by post-hoc Duncan-test. The percentage change in the horizontal activity, immobility or “FST-activity” variables were calculated for each dose according to the equation;

%=[(X−Y)/Y]×100

where X is the mean value for the drug-treated group and Y is the mean value for the vehicle-treated group. The results are shown in Table 1.

TABLE 1

| | Spontaneous | | | | | |
|---|---|---|---|---|---|---|
| | Locomotor Activity | | | | | |
| | No. of Horizontal | | Forced Swimming Test | | | |
| | Movements | | Immobility Time | | “FST-Activity” | |
| | Sum | % | Sum | % | Sum | % |
| | (60 mins) | Increase | (sec) | Inhibition | (sec) | Increase |
| Vehicle | 1837.8 ± 238.92 | — | 218.0 ± 10.40 | — | 142.0 ± 10.40 | — |
| (DIMEB-80 p.o) | | | | | | |
| Radiprodil/DIMEB | 2149.4 ± 211.62 | 17 | 199.4 ± 10.80 | 9 | 160.6 ± 10.80 | 13 |
| (2.5 mg/kg) | | | | | | |
| Radiprodil/DIMEB | 2897.2 ± 321.62* | 58 | 152.4 ± 14.43** | 12 | 207.6 ± 14.43** | 46 |
| (5 mg/kg) | | | | | | |
| Radiprodil/DIMEB | 4409.3 ± 324.91*** | 140 | 113.9 ± 13.85*** | 48 | 246.1 ± 13.85*** | 73 |
| (10 mg/kg) | | | | | | |

*p < 0.05,
**p < 0.01,
***p < 0.001 compared to the control group

As can be seen from Table 1, radiprodil showed significant effects in the forced swimming test at doses of 5 and 10 mg/kg. Radiprodil also stimulated the spontaneous motor activity of the mice at the same doses. The effect of radiprodil in the forced swimming test may have resulted from a nonspecific stimulatory effect and may be considered a false positive result. Results from assays insensitive to changes in motor activity may, however, surprisingly show that radiprodil can be used to safely and effectively treat depression.

### Example 2

This study will determine the effects of radiprodil administration on behavioral impairment in triple transgenic mice that act as a model for Alzheimer's disease in humans (3xTg-AD mice) by using well-characterized behavioral assays that are designed to identify deficits in spatial memory, object recognition and fear conditioning.

### Design and Methods

Mice: 3xTg-AD mice with a hemizygous (PS1_{M146V}/PS1_{M146V}; APP_{Swe}+/0; Tau_{P301L}+/0) and homozygous (PS1_{M146v}/PS1_{M146V}; APP_{Swe}+/+; Tau_{P301L}+/+) genotype will be used. The mice develop both plaques and tangles in a hierarchical, region specific and age-progressive manner that mimics the development of Alzheimer's disease in humans. See Oddo et al., Neuron, 39(3), 409-421, 2003. This study will focus on behavioral changes in 3xTg-AD (homoz) mice administered radiprodil. Age- and sex-matched non-transgenic (NonTg) mice will also be included as a control group.

Subjects and drug administration: 9, 12 and 15-month-old mice will be treated for 3 months with radiprodil. For statistical purposes each group will consist of 10 mice, plus 5 mice in the oldest 3xTg-AD group. Mice will be tested on all tasks at the end of the study. Therefore, there will be 40 or 50 animals per each time point (10 animals per group×2 genotypes×2 treatment groups. The proposed study groups are shown in Table 2.

TABLE 2
Proposed Study Groups

| | 6-9 months | 9-12 months | 15-18 months |
|---|---|---|---|
| | Age of 3xTg-AD mice | | |
| Control group | 10 | 10 | 15 |
| Radiprodil | 10 | 10 | 15 |
| | Age of NonTg mice | | |
| Control group | 10 | 10 | 15 |
| Radiprodil | 10 | 10 | 15 |
| Total number of animals | 40 | 40 | 60 |
| in study = 140 | | | |

All animals will be given free access to food and water. All animals will be sacrificed after their 3 month testing. A detailed neuropathological, histochemical and biochemical analysis of the brain will be performed to determine Aβ loads and the extent of tau pathology.

### Biochemical Markers

Aβ Measurements:

Quantitative data will be obtained on the effects of radiprodil on various species of Aβ (e.g. Aβ40 versus Aβ42; soluble versus insoluble Aβ). Protein extracted from brain tissue from mice treated with radiprodil will be used to generate soluble and insoluble protein extracts and analyzed by sandwich ELISA. Western blots will also be performed to measure steady state levels of the APP holoprotein, C99 fragment, and APP secreted to determine the effects of radiprodil on these biomarkers.

Tau Hyperphosphorylation:

The effects of radiprodil on tau hyperphosphorylation as a functional biomarker will be evaluated using quantitative western blotting with antibodies (such as AT8, AT100, or PHF1) that specifically recognize hyperphosphorylated tau.

### Behavioral Assays

Morris Water Maze

Spatial memory will be tested on the Morris Water Maze (MWM). The water maze is a circular pool (diameter=1 meter) filled with water maintained at 26° C. and made opaque by the addition of powdered milk. Mice will be pre-trained by swimming to a 12×12 cm poly(methyl methacrylate) platform submerged 1.5 cm beneath the surface of the water. The location of the platform will be selected randomly for each mouse, but will be kept in a constant position for each individual mouse throughout training. The maze will be located in a room containing several visual, extra-maze cues. For spatial training, mice will be subjected to four trials a day for as many days as required to reach criterion. Before the first training trial, the mouse will be placed on the platform for 10 seconds. On each trial (swim), the mouse will be placed into the tank at one of four designated start points in a random order. Mice will be allowed to find and escape onto the submerged platform. If an animal fails to find the platform within 60 seconds, it will be manually guided to the platform and will remain there for 5 seconds. After this, each mouse will be placed into a holding cage under a warming lamp for 25 seconds until the start of the next trial.

Retention of the spatial training will be assessed 1.5 hours and again 24 hours after the last training trial. Both of these probe trials will consist of a 60 second free swim in the pool with the platform removed. Mice will be monitored by a camera mounted in the ceiling directly above the pool, and all trials will be stored on videotape for subsequent analysis. The parameters measured during the probe trial will include (1) time spent in the quadrant opposite to the quadrant containing the platform during training and (2) initial latency to cross the platform location and (3) number of crosses of platform location. For each time point the target quadrant will vary for each animal to avoid “savings” from previous water maze experience. At each time point, target quadrants will vary between animals within a group to control for potential differences in the salience of extramaze cues.

The escape data will be examined with a multifactor analysis of variance (ANOVA) including genotype (transgenic vs. control), treatment (radiprodil vs. control) and probe trial (1.5 or 24 hours). Post-hoc tests will determine individual differences in groups with respect to controls (nontransgenic mice) for each time point.

Object Recognition Task

This task is based on the spontaneous tendency of rodents to explore a novel object more often than a familiar object and is widely used to study memory impairments in AD models. See, e.g., Ennaceur et al., Behav. Brain Res., 31(1), 47-59, 1988; Dodart et al., Nat. Neurosci., 5(5), 452-457, 2002; Vaucher et al., Exp. Neurol., 175(2), 298-406, 2002.

On the first day of testing, mice will be subjected to a 5 minute familiarization session in the empty open field. On the next day, mice will be subjected to a 5-minute exploration session in the same open field with two identical objects (Object A; e.g. two identical marbles or two identical dice) placed in symmetrical locations in the open field. 15 minutes and 24 hours later, animals will be subjected to a 5-minute retention phase where they will again be exposed to one Object A and also to a novel object, Object B (for the 15 minute time point) and Object C (for the 24 hour time point) placed in the same, symmetrical locations in the open field. The time spent exploring the familiar object (Object A) and the novel object (Object B or C) will be calculated where exploration equals touching the object with nose or paws, or sniffing within 1.5 cm of the object. A memory index (MI) will be calculated such that MI=(tn−tf)/(tn+tf) where tf is the time spent exploring the familiar object (Object A) and to is the time spent exploring the novel object (Object B or C). This memory index will provide a score that represents the relative amount of time spent exploring the familiar versus novel object. All objects and the open field will be wiped down with 70% ethanol after each trial to remove olfactory-related cues.

Differences in the MI score will be analyzed using a multifactor ANOVA including genotype, treatment group, age and probe trial (15 minutes and 24 hours). Post-hoc tests will determine individual differences in groups with respect to controls (nontransgenic mice) for each time point.

### Inhibitory-Avoidance Procedure

Inhibitory avoidance will be measured using the Gemini Avoidance System with the grid floor designed for mice (San Diego Instruments, San Diego, Calif.). This apparatus consists of two chambers, a light and dark compartment (each 25.4×20.3 cm). A door (8.9×8.9 cm) separates the two compartments. The procedure consists of a training trial and a retention trial. In the training trial, mice will be placed in the lighted compartment of the inhibitory avoidance box. After a mouse enters the dark compartment, the door between the two compartments will be closed and the latency to enter the dark compartment will be recorded (baseline latency). After the door closes, the mouse will immediately be given a 0.3-0.5 mA footshock (1 second duration; the size of the footshock will be determined by pilot studies to avoid ceiling or floor retention latencies). The animal will remain in the dark compartment for an additional 10 seconds before being returned to the home cage. Retention trials will be conducted 1.5 hours and 24 hours after the training trial. During the retention trial, the mouse will again be placed in the lighted compartment and the latency to enter the dark compartment will be recorded. The maximum amount of time allowed to enter the dark compartment will be 180 seconds. For testing at each time point after the 2 month time point, an initial retention trial will be conducted before fear conditioning is repeated.

Difference in latency scores will analyzed by a multifactor ANOVA including genotype (transgenic vs. control), treatment (radiprodil vs. control) and probe trial (1.5 or 24 hours). Post-hoc tests will determine individual differences in groups with respect to controls (nontransgenic mice) for each time point.

The results from the above treatment regimes may surprisingly show that radiprodil can be used to safely and effectively treat Alzheimer's disease.

### Example 3

A patient with Alzheimer's disease presents to a physician's office or clinic. To improve the patient's symptoms, the patient is administered between about 1 and about 150 mg radiprodil per day. The patient's vital signs and an ECG are recorded. Adverse events are also recorded. Physical examinations are conducted and blood and urine samples are collected. At the discretion of the physician, the dosage of radiprodil can be reduced or increased as required. The results from the above treatment regimen may surprisingly show that radiprodil can be used to safely and effectively treat Alzheimer's disease.

### Example 4

A patient with major depressive disorder presents to a physician's office or clinic. To improve the patient's symptoms, the patient is administered between about 1 and about 150 mg radiprodil per day. The patient's vital signs and an ECG are recorded. Adverse events are also recorded. Physical examinations are conducted and blood and urine samples are collected. At the discretion of the physician, the dosage of radiprodil can be reduced or increased as required. The results from the above treatment regimen may surprisingly show that radiprodil can be used to safely and effectively treat major depressive disorder.

### Example 5

A patient with anxiety presents to a physician's office or clinic. To improve the patient's symptoms, the patient is administered between about 1 and about 150 mg radiprodil per day. The patient's vital signs and an ECG are recorded. Adverse events are also recorded. Physical examinations are conducted and blood and urine samples are collected. At the discretion of the physician, the dosage of radiprodil can be reduced or increased as required. The results from the above treatment regimen may surprisingly show that radiprodil can be used to safely and effectively treat anxiety.

### Example 6

A patient with major depressive disorder presents to a physician's office or clinic. To improve the patient's symptoms, the patient is administered a combination of radiprodil and escitalopram oxalate. The patient's vital signs and an ECG are recorded. Adverse events are also recorded. Physical examinations are conducted and blood and urine samples are collected. At the discretion of the physician, the dosage of radiprodil and/or escitalopram oxalate can be reduced or increased as required. The results from the above treatment regimen may surprisingly show that a combination of radiprodil and escitalopram oxalate can be used to safely and effectively treat major depressive disorder. The combination of radiprodil and escitalopram oxalate may provide synergistic benefit when compared to patients treated with radiprodil or escitalopram oxalate alone.

### Example 7

A patient with Alzheimer's disease presents to a physician's office or clinic. To improve the patient's symptoms, the patient is administered a combination of radiprodil and memantine hydrochloride. The patient's vital signs and an ECG are recorded. Adverse events are also recorded. Physical examinations are conducted and blood and urine samples are collected. At the discretion of the physician, the dosage of radiprodil and/or memantine hydrochloride can be reduced or increased as required. The results from the above treatment regimen may surprisingly show that a combination of radiprodil and memantine hydrochloride can be used to safely and effectively treat Alzheimer's disease. The combination of radiprodil and memantine hydrochloride may provide synergistic benefit when compared to patients treated with radiprodil or memantine hydrochloride alone.

### Example 8

A patient suffering from depression presents to a physician's office or clinic. To improve the patient's symptoms, the patient is administered a combination of radiprodil and milnacipran hydrochloride. The patient's vital signs and an ECG are recorded. Adverse events are also recorded. Physical examinations are conducted and blood and urine samples are collected. At the discretion of the physician, the dosage of radiprodil and/or milnacipran hydrochloride can be reduced or increased as required. The results from the above treatment regimen may surprisingly show that a combination of radiprodil and milnacipran hydrochloride can be used to safely and effectively treat depression. The combination of radiprodil and milnacipran hydrochloride may provide synergistic benefit when compared to patients treated with radiprodil or milnacipran hydrochloride alone.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

The entire disclosures of all applications, patents and publications cited herein are hereby incorporated by reference in their entirety.

What is claimed is:

1. A method of treating a disorder selected from Alzheimer's disease, anxiety and major depressive disorder comprising administering to a patient in need thereof a therapeutically effective amount of a compound of formula (I): 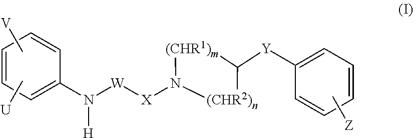
wherein
V and U are each independently
hydrogen, halogen, hydroxyl, cyano, nitro, amino, C₁-C₄ alkylamino optionally substituted by one or more halogen, arylamino optionally substituted by one or more halogen, aralkylamino optionally substituted by one or more halogen, C₁-C₄ alkylsulfonamido optionally substituted by one or more halogen, C₁-C₄ alkanoylamido optionally substituted by one or more halogen, arylsulfonamido, alkylsulfonyloxy, carboxyl, trifluoromethyl, trifluoromethoxy, C₁-C₄ alkyl-SO₂—NH—CH₂—, NH₂—(CH₂)₁₋₄—SO₂—NH—, NH₂—(CH₂)₁₋₄—(CO)—NH—, sulfamoyl, formyl, aminomethyl, hydroxymethyl, C₁-C₄ alkyl, C₁-C₄ alkoxymethyl, halogenated methyl, tetrazolyl,
or C₁-C₄ alkoxy, alkoxycarbonyl, C₁-C₆ alkanoyloxy, phenyl or C₁-C₄ alkoxy, each of which is optionally substituted by an amino group, or
neighboring V and U groups, together with one or more identical or different additional heteroatoms and/or —CH=and/or —CH₂— groups optionally form a substituted 4-7 membered homo- or heterocyclic ring;
W and X are each independently —CO—, —CH₂— or —CH(C₁-C4 alkyl)-, with the proviso that W and X can not simultaneously be methylene;
Y is —O—, C₁-C₄ alkylene, C₁-C₄ alkynylene, cycloalkylene, aminocarbonyl, —NH—, —N(C₁-C₄ alkyl)-, —CH₂O—, —CH(OH)— or —OCH₂—;
Z is hydrogen, halogen, nitro, amino, C₁-C₄ alkyl, C₁-C₄ alkoxy, cyano, trifluoromethyl, hydroxyl or carboxy;
R¹ and R² are each independently hydrogen or alkyl, or R¹ and R² together form an optionally substituted C₁-C₃ bridge and
n and m independently are 0-3, with the proviso that n and m can not simultaneously be 0;
and pharmaceutically acceptable salts or solvates (e.g., hydrates) thereof, or solvates of pharmaceutically acceptable salts thereof;
with the further provisos that
when Z is hydrogen, Y is —CH₂—, m and n are 2, R¹ and R² are hydrogen, W is —CO—, X is —CH₂— and V is hydrogen, then U is other than a 4-bromo substituent, and
when Z is hydrogen, Y is —CH₂—, m and n are 2, R¹ and R² are hydrogen, W and X are —CO— and V is hydrogen, then U is other than a 4-carboxyl or 4-ethoxycarbonyl substituent.

2. The method according to claim 1, wherein the compound of formula (I) is 2-[4-(4-fluoro-benzyl)-piperidine-1-yl]-2-oxo-N-(2-oxo-2,3-dihydro-benzoxazol-6-yl)acetamide, or a pharmaceutically acceptable salt thereof, solvate thereof, or a solvate of a pharmaceutically acceptable salt thereof.

3. The method according to claim 2, wherein the disorder is Alzheimer's disease.

4. The method according to claim 2, wherein the disorder is major depressive disorder.

5. The method according to claim 2, wherein the disorder is anxiety.

6. The method according to claim 2, wherein therapeutically effective amount administered is from about 10 mg to about 150 mg.

7. The method according to claim 6, wherein the compound of formula (I) is administered in one, two, three or four divided daily doses.

8. The method according to claim 3, wherein therapeutically effective amount administered is from about 10 mg to about 150 mg.

9. The method according to claim 4, wherein therapeutically effective amount administered is from about 10 mg to about 150 mg.

10. The method according to claim 5, wherein therapeutically effective amount administered is from about 10 mg to about 150 mg.

11. The method according to claim 1, wherein the compound of formula (I) is adjunctively administered with a tricyclic antidepressant, selective serotonin reuptake inhibitor, norepinephrine reuptake inhibitor, norepinephrine-dopamine reuptake inhibitor, serotonin-norepinephrine reuptake inhibitor, monoamine oxidase inhibitor, cholinesterase inhibitor and combinations thereof

12. The method of claim 1, wherein the compound of formula (I) is adjunctively administered with memantine, escitalopram, citalopram, milnacipran, donezepil, rivastigmine, galantamine, fluvoxamine, paroxetine, reboxetine, sertraline, amitriptyline, desipramine, nortriptyline, duloxetine, venlafaxine, mirtazepine, trazodone, bupropion and combinations thereof.
